SEQUENCE LISTING (8 sequences)

SEQ ID NO: 1 - DNA, 33 bp, T. thermophila
aattcgtaatacgactcactataggttctatag

SEQ ID NO: 2 - DNA, 25 bp, T. thermophila
agaacctatagtgagtcgtattacg

SEQ ID NO: 3 - DNA, 28 bp, T. thermophila
tatagcggttagtactggggactctaaa

SEQ ID NO: 4 - DNA, 28 bp, T. thermophila
gagtccccagtactaaccgctatactat

SEQ ID NO: 5 - DNA, 18 bp, T. thermophila
tcccttgacctgggttcg

SEQ ID NO: 6 - DNA, 18 bp, T. thermophila
cccaggtcaagggattta

SEQ ID NO: 7 - DNA, 32 bp, T. thermophila
aatcccagtaggaccgccatgagacccatccg

SEQ ID NO: 8 - DNA, 39 bp, T. thermophila
gatccggatgggtctcatggcggtcctactgggattcga

(19) United States
(12) Patent Application Publication
(10) Pub. No.: US 2004/0180401 A1
(43) Pub. Date: Sep. 16, 2004

(54) Methods of determining precise HERG interactions and designing compounds based on said interactions

(75) Inventors: Dennis A. Dougherty, South Pasadena, CA (US); Henry A. Lester, Pasadena, CA (US); Jonathan G. Lasch, Pasadena, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Neurion Pharmaceuticals, Inc., Pasadena, CA (US)

(21) Appl. No.: 10/444,058

(22) Filed: May 23, 2003

Related U.S. Application Data
(60) Provisional application No. 60/382,571, filed on May 24, 2002.
(60) Provisional application No. 60/454,338, filed on Mar. 14, 2003.

(51) Int. Cl. C12Q 1/68; C07H 21/04; C07K 14/705

(52) U.S. Cl. 435/69.1; 435/320.1; 435/325; 530/350; 536/23.5; 548/304.1; 548/493; 562/444; 562/507

(57) ABSTRACT

The present invention discloses methods of determining highly precise interactions between the HERG ion channel and various compounds. The methods of the present invention utilize the nonsense codon suppression methods combined with heterologous in vivo expression in Xenopus oocytes.

This application is related to co-assigned U.S. patent application No. 60/382,571, filed May 24, 2002, entitled “Methods of Determining Precise HERG Interactions and Designing Compounds Based on Said Interactions”, and U.S. patent application No. 60/454,338, filed Mar. 14, 2003, also entitled “Methods of Determining Precise HERG Interactions and Designing Compounds Based on Said Interactions” the entireties of which are incorporated by reference herein.

### FIELD OF THE INVENTION

The present invention generally relates to methods of obtaining high-precision structural and functional information on the membrane protein ion channel HERG. The present invention more specifically relates to methods using nonsense codon suppression and in vivo and heterologous expression, which enable determination of HERG binding by compounds to a very high specificity. Unexpected HERG activity, i.e. non-specific modulatory effects, limits the efficacy of many drugs, and can even cause dangerous side effects. The present invention also relates to methods for the discovery and design of safer and more selective compounds without unexpected HERG activity.

### BACKGROUND OF THE INVENTION

Voltage-gated potassium channels are key determinants of normal cellular activity, but can contribute to disease and, consequently, are increasingly recognized as potential therapeutic targets. Changes in the properties of potassium channels and even the types expressed have been linked to several cardiac and neurological diseases. Nerbonne (1998) J. Neurobiol. 37:37-59.

The human ether-à-go-go related gene (hereinafter, HERG) K⁺ channel is one of the myriad of ion channels responsible for generating the cardiac action potential. HERG encodes an inwardly-rectifying potassium channel that plays an important role in repolarization of the cardiac action potential. Inward rectification of HERG channels results from rapid and voltage-dependent inactivation gating, combined with very slow activation gating.

HERG was originally cloned from human hippocampus by Warmke et al. ((1994) Proc. Natl. Acad. Sci USA 91:3438-3442, and is strongly expressed in the heart. The hydropathy plot for the HERG protein suggests that this channel resembles the Shaker potassium channel; both have a six transmembrane region subunit structure with a highly charged fourth transmembrane segment. Despite this similarity, HERG channels behave very differently from Shaker channels: HERG behaves like an inward rectifier rather than an outward rectifier. Sanguinetti et al.(1995) Cell 81:299-307. This anomalous behavior is due to the unusual kinetics of HERG gating, with slow activation gating and fast inactivation gating. During depolarization, HERG channels slowly activate and then rapidly inactivate, resulting in little outward current; during subsequent hyperpolarization, channels recover rapidly from inactivation but deactivate slowly, resulting in a large inward current.

Long QT syndrome (LQT) is an abnormality of cardiac muscle repolarization that predisposes affected individuals to a ventricular arrhythmia that can degenerate into ventricular fibrillation and cause sudden death. The HERG ion channel has been linked to QT interval prolongation and sudden death. Mutations in the HERG channel gene cause inherited long QT. However, QT interval prolongation can also be caused by non-genetic, or extrinsic causes. In recent years, several prescription drugs have been speculated to be responsible for this QT interval prolongation, and therefore linked to HERG activity. Drugs such as Seldane, Propulsid, Hismanal, and others have been removed from the market because of their potential cardiac side effects and suspected HERG activity. Additionally, many promising drugs in clinical trials and countless pre-clinical compounds have been removed from the development pathway because of activity at the HERG ion channel. This has led to literally billions of dollars of lost revenues and sunk development costs.

Unexpected HERG activity, whether for inherited or non-inherited reasons, has been an area of increasing frustration for the pharmaceutical industry. The FDA now recommends that pharmaceutical companies have detailed in vitro and in vivo pre-clinical tests to screen for potentially hazardous compounds that prolong the QT interval on ECG readings (“ICH Guideline on Safety Pharmacology Studies for Human Pharmaceuticals” (ICH S7A), Feb. 7, 2002).

Therefore, methods of determining this unexpected activity are highly desirable to the pharmaceutical industry. Methods of nonsense codon suppression have been used to probe structure-function relationships in receptor binding sites of other channels. Nowak et al (1995) Science 268:439. This method of combining site-directed mutagenesis and heterologous expression was instrumental in elucidating the functional relationships of the nicotinic receptor with its agonists and antagonists. Id. Application of these methods to the HERG system may help elucidate and possibly control the unexpected activity that leads to prolonged QT intervals.

Current HERG screening reveals information about the existence and strength of HERG binding, but does not give precise details on the nature and location of the binding, and or instructions about how one could make subtle modifications to compounds in order to avoid HERG activity. The present invention will not only provide information on whether a compound binds to HERG, but also details both the method and specific location of binding. Through high-precision compound modifications, the present invention will enable the identification and continued development of drug classes that would otherwise be dropped because of HERG activity, or make compounds to block and reduce the HERG activity of other compounds as adjuvants.

### SUMMARY OF THE INVENTION

Methods of determining precise compound interactions with the HERG ion channel are disclosed. More specifically, methods of incorporating unnatural amino acids into HERG ion channels expressed in intact cells are provided, so that structure-function relationships may be probed. Furthermore, high-precision methods of determining HERG interactions are disclosed herein.

An object of the invention is to provide a method of incorporating unnatural amino acids into the HERG ion channel comprising: a) determining sites of potential antagonist or agonist interaction with the HERG ion channel; b) using the nonsense codon suppression method to incorporate unnatural amino acids into the sites determined in (a); and c) determining binding interactions of the compound of interest with the HERG ion channel.

It is a further object of the invention to provide a systematic method of determining the nature of a compound's interaction with HERG comprising: a) incorporating unnatural amino acids into binding and regulatory sites of HERG, resulting in an altered HERG; b) measuring the compound's ability to bind to the altered HERG; and c) comparing the results of step (b) to the same compound's ability to bind to an unaltered HERG.

It is yet a further object of the invention to provide a systematic method of screening for compounds which cause cardiac toxicity comprising developing an assay system, wherein said system allows for a) searching of compounds that prolong QT interval on ECG readings, then b) using said system to determine details of the nature and location of HERG binding of said compounds; and finally c) determining which compounds are causing said toxicity by evaluating how and where said compound binds to HERG.

It is another object of the invention to provide a receptophore model, which provides a 3-dimensional picture of compounds contact points at the HERG channel binding sites.

It is also an object of the invention to provide a method of altering a compound so that it does not interact with HERG comprising: a) determining the nature of the compound's interaction with HERG; b) analyzing how and where the compound interacts with HERG; based on the analysis in step (b), and c) chemically modifying the compound to avoid HERG interaction.

It is another object of the invention to provide a methods of designing compounds that will inhibit, hinder, or block other compounds from unfavorable HERG interactions. This may allow for attenuation of compounds with HERG activity.

Another object of the invention to provide a HERG screening assay system comprising a HERG channel which has been modified to replace native amino acids with unnatural amino acids, wherein the channel is expressed in vivo in Xenopus oocytes.

### BRIEF DESCRIPTION OF THE DRAWINGS

FIG. 1. Scheme for incorporating unnatural amino acids into proteins expressed in Xenopus oocytes.

FIG. 2. Plot of log[EC₅₀/EC_{50(WT)}] vs. cation-π binding ability at α-Trp149 of the nicotinic acetylcholine receptor for the wild type trp and the fluorinated trp derivatives 5-F-Trp, 5,7-F₂-Trp, 5,6,7-F₃-Trp, and 4,5,6,7-F₄-Trp.

### DETAILED DESCRIPTION OF THE INVENTION

The present invention describes a method of obtaining highly precise binding and interaction information of ligands or drugs with the HERG ion channel by utilizing incorporation of unnatural amino acids at critical sites within the transmembrane domains of the ion channel. The information elucidated from these novel experiments will allow predictive identification of binding molecules or drugs that contribute or cause undesirable HERG activity as well as ones that alleviate such activity.

As used herein, the term “HERG” means the human ether-à-go-go related potassium ion channel, which has 6 transmembrane chains. This HERG polypeptide exhibits structural similarities to members of the S4-containing superfamily of ion channels and its behavior can be described by typical gating characteristics, such as sigmoidal time course of activation and C-type inactivation.

As used herein, a Voltage-Gated Ion channel (VGIC) represents a group of cell membrane channel proteins. These proteins of the VGIC family are ion-selective channel proteins found in a wide range of bacteria, archaea and eukaryotes. Functionally characterized members are specific for K⁺, Na⁺ or Ca²⁺. The K⁺ channels usually consist of homotetrameric structures with each subunit possessing six transmembrane spanners (TMSs). Many voltage-sensitive K⁺ channels function with subunits that modify K⁺ channel gating. Some of these auxiliary subunits, but not those of a HERG channel, are oxidoreductases that coassemble with the tetrameric subunits in the endoplasmic reticulum and remain tightly adherent to the subunit tetramer. High resolution structures of some potassium channels, but not of HERG channels are available (e.g. Jiang et al., Nature (2002) May 30;417(6888):515-22). The high resolution structure of a beta subunit is available (Gulbis et al., Cell (1999) June 25;97(7):943-52).

In eukaryotes, each VGIC family channel type has several subtypes based on pharmacological and electrophysiological data. Thus, there are five types of Ca²⁺ channels (L, N, P, Q and T). There are at least ten types of K⁺ channels, each responding in different ways to different stimuli: voltage-sensitive [Ka, Kv, Kvr, Kvs and Ksr], Ca²⁺-sensitive [BK_{Ca}, IK_{Ca} and SK_{Ca}], and receptor-coupled [K_{M} and K_{ACh}]. There are at least six types of Na⁺ channels (I, II, III, μl, H1 and PN3). Tetrameric channels from both prokaryotic and eukaryotic organisms are known in which each subunit possesses 2 TMSs rather than 6, and these two TMSs are homologous to, TMSs 5 and 6 of the six TMS units found in the voltage-sensitive channel proteins. The KcsA of S. lividans is an example of such a 2 TMS channel protein. These channels may include the K_{Na} (Na⁺-activated) and K_{Vol}(cell volume-sensitive) K⁺0 channels, as well as distantly related channels such as the Tokl K⁺ channel of yeast. The TWIK-1 and -2, TREK-1, TRAAK, and TASK-1 and -2 K⁺ channels all exhibit a duplicated 2 TMS unit and may therefore form a homodimeric channel. About 50 of these 4 TMS proteins are encoded in the C. elegans genome. Because of insufficient sequence similarity with proteins of the VGIC family, inward rectifier K⁺ IRK channels (ATP-regulated or G-protein-activated), which possess a P domain and two flanking TMSs, are placed in a distinct family (TC #1.A.2). However, substantial sequence similarity in the P region suggests that they are homologous. The subunits of VGIC family members, when present, frequently play regulatory roles in channel activation/deactivation.

As used herein, the HERG assay measures the modified HERG ion channel, as modified with unnatural amino acids and expressed in Xenopus oocytes as it interacts with chemical entities of interest.

The receptophore model, as used herein, is the ensemble of steric and electronic features of a biological target that are necessary to ensure optimal supramolecular interactions with a specific ligand and to trigger (or block) the biological function of the target.

The QT interval as used herein is the time period it takes for cardiac repolarization as measured on an electrocardiogram. Prolongation of this interval can lead to generation of the life threatening ventricular arrhythmia known as torsades de pointes. Ben-Davies et al. (1993) Lancet 341:1578. Similarly, the long QT syndrome is an abnormality of cardiac muscle repolarization that predisposes affected individuals to a ventricular arrhythmia that can degenerate into ventricular fibrillation and cause sudden death.

As used herein, the electrocardiogram (hereinafter, “ECG”) is a common test for measuring detailed heart rhythms, waves, and beats.

As used herein, an “unnatural amino acid” is any amino acid other than one of the 20 recognized natural amino acids as provided in Creighton, Proteins, (W.H. Freeman and Co. 1984) pp.2-53.

HERG Structure and Function

The HERG ion channel is a member of the depolarization-activated potassium channel family, which has 6 putative transmembrane spanning domains. This is unusual because the ion channel exhibits rectification like that of the inward-rectifying potassium channels, which only have 2 transmembrane domains. Smith et al. (1996) Nature 379:833, studied HERG channels expressed in mammalian cells and found that this inward rectification arises from a rapid, voltage-dependent inactivation process that reduces conductance at positive voltages. The inactivation gating mechanism of HERG resembles that of C-type inactivation, often considered to be the ‘slow’ inactivation mechanism of other potassium channels. Characteristics of this gating suggested a specific role for this channel in the normal suppression of arrhythmias. The role for HERG in suppressing extra beats might help explain the increased incidence of cardiac sudden death in patients that lack HERG currents, either because they carry a genetic defect or because for example they are being treated with class III antiarrhythmics that block HERG channels. Therefore, determination of binding interaction of any drug or compound of this type with the HERG channel would provide information on how to avoid this interaction.

Crystallization is one conventional method for studying three-dimensional structures and their interaction with drug compounds. However, elucidation of a crystal structure is very time-consuming, and the results are not always precise enough to determine all the possible interactions. In case of membrane proteins (i.e. HERG ion channel), numerous attempts have failed at co-crystallizing the proteins with various known channel blockers in attempts to study the binding site interactions. Additionally, given the dynamic nature of the HERG channel, a static crystal picture may not be in the proper functional context. Lastly, conformation of the protein under investigation may be altered due to crystal packing forces. The methods described herein provide highly precise interaction and binding data without crystallography. In the absence of atomic-scale structural data for membrane proteins such as that provided by crystallography, these techniques can provide detailed structural information.

To determine which sites on the HERG ion channel to modify using the inventive methods, it is helpful to look at previous studies with the HERG ion channel. For example, conventional mutagenesis studies of the HERG ion channel can provide information on possible binding sites within the transmembrane domains. See Mitcheson et al. (2002) Proc. Natl. Acad. Sci. 97:12329-12333. The inner cavity of the HERG channel may be much larger than any other voltage-gated potassium channel, based on sequence analysis and comparison with the KcsA homology model. Also unlike other voltage-gated potassium channels, the S6 domains of the HERG channels have two aromatic residues that face into the inner cavity. These residues, among others, may bind drugs, leading to the unexpected HERG activity. Previously, it has been reported that the binding site of HERG is comprised of amino acids located on the S6 transmembrane domain (G648, Y652, and F656) and pore helix (T623 and V625). See Mitcheson et al. Therefore, these sites are preferred for incorporation of the unnatural amino acids.

Generation of Receptophore Model

An accurate receptophore model is built through identification of amino acids involved in the ligand binding site and the probing of the molecular forces involved. First, unnatural amino acids are incorporated into the HERG ion channel using nonsense suppression methodology. Altered ion channels are expressed heterologously on Xenopus oocyte membranes. Compounds are screened for binding efficacy to the altered channel. Electrophysiological or biochemical assays are used to measure the effects, if any, of unnatural amino acid substitutions on ligand binding. Binding data involving the wild-type versus the altered channel are compared to define the molecular forces involved in ligand binding.

The interaction of acetylcholine with the nicotinic acetylcholine receptor has recently been studied in order to develop the receptophore model for the interactions of the nicotinic agonists described in Zhong et al. (1998) Proc. Natl. Acad. Sci. 95:12088-12093. A clear agonist receptophore model of the nicotinic receptor family will emerge after multiple agonist contact points are identified through systematic mapping of the target binding sites using the in vivo nonsense suppression method for unnatural amino acid incorporation. A number of aromatic amino acids have been identified as contributing to the agonist binding site, suggesting that cation-p interactions may be involved in binding the quaternary ammonium group of the agonist, acetylcholine. A compelling correlation has been shown between (i) ab initio quantum mechanical predictions of cation-p binding abilities and (ii) EC₅₀ values for acetylcholine at the receptor for a series of tryptophan derivatives that were incorporated into the receptor by using in vivo nonsense suppression method for unnatural amino acid incorporation. Such a correlation is seen at one, and only one, of the aromatic residues: tryptophan-149 of the a subunit. This finding indicates that, on binding, the cationic, quaternary ammonium group of acetylcholine makes van der Waals contact with the indole side chain of the a tryptophan-149, providing the most precise structural information to date on this receptor. Upon similar systematic probing of other potential steric and electronic interactions at the acetylcholine binding site, a receptophore model will be built for binding and physiological activity of agonists at the nicotinic receptor. This general methodology can be used to build receptophore models for other agonist, antagonist, or allosteric interactions with a wide range of receptors and ion channels. Id.

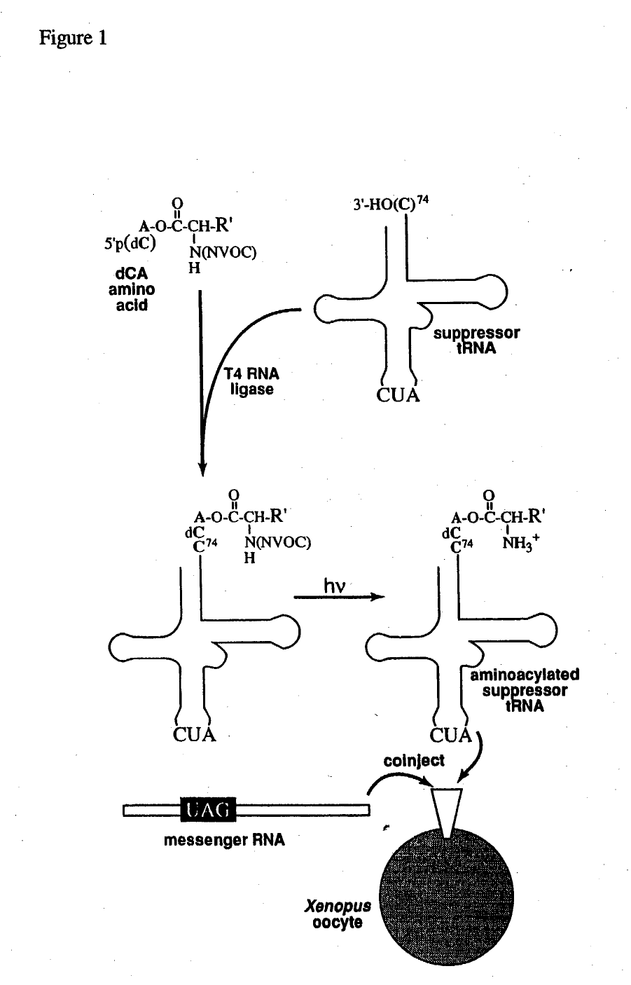

Unnatural amino acids are incorporated into the HERG ion channel binding sites through the use of nonsense codon suppression. Noren et al. (1989) Science 244:182; Nowak et al. (1998) Methods in Enzymol. 293:515. See FIG. 1. In the nonsense suppression method, two RNA species are prepared using standard techniques such as in vitro synthesis from linearized plasmids. The first is an mRNA encoding the HERG channel but engineered to contain an amber stop codon (UAG) at the position where unnatural amino acid incorporation is desired. The second is a suppressor tRNA that contains the corresponding anticodon (CUA) and that is compatible with the expression system employed, such as Tetrahymena thermophila tRNA^{Gln} G73 for Xenopus oocytes or E. coli expression systems. The tRNA is then chemically acylated at the 3′ end with the desired unnatural amino acid using techniques known in the art such as that described in Kearney et al. (1996), Mol. Pharmacol., 50: 1401-1412.

Synthesis of the unnatural amino acids depends on the desired structure. The unnatural amino acid may be prepared, for example, by modification of a natural amino acid. Also, many unnatural amino acids are commercially available. The NRC Biotechnology Research Institute Peptide/Protein Chemistry Group maintains an excellent listing of commercially available amino acids at http://aminoacid.bri.nrc.ca.

Examples of preferred unnatural amino acids for incorporation into mammalian cells using the methods of the present invention include, but are not limited to, those represented by the following Formula (I):

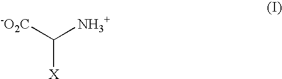

where X is selected from the group consisting of:

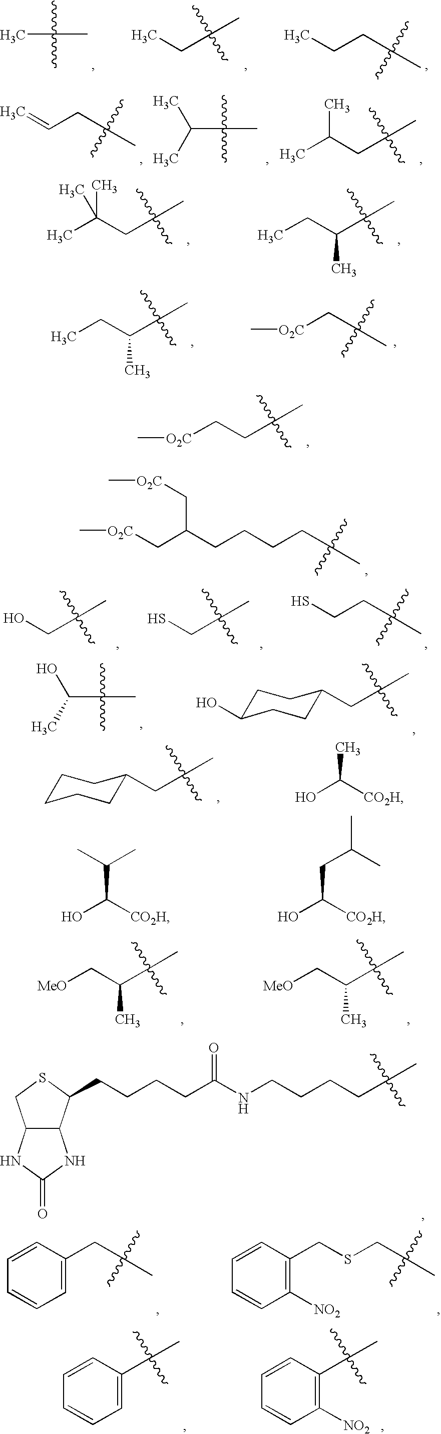

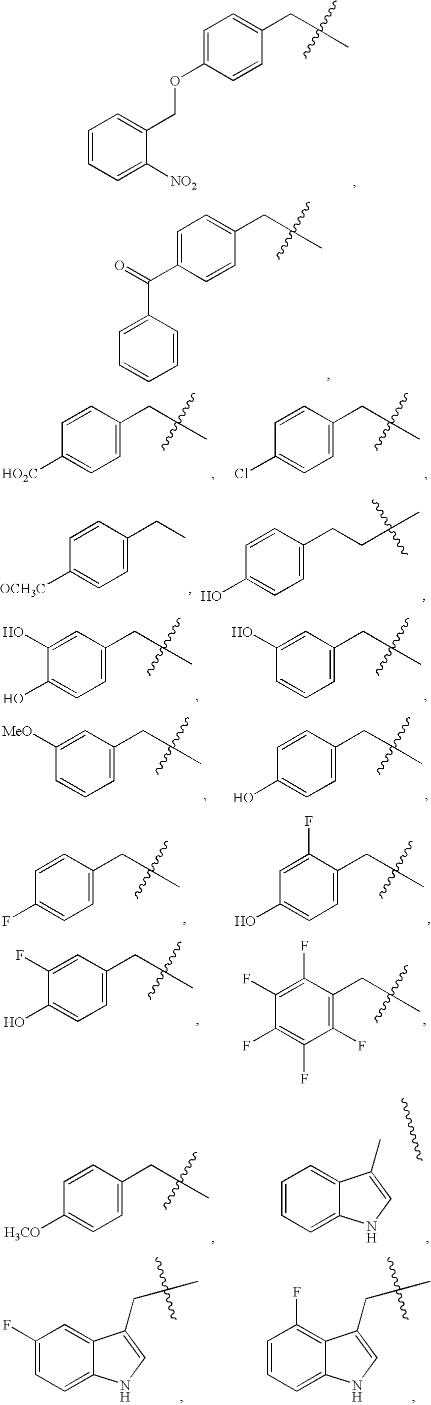

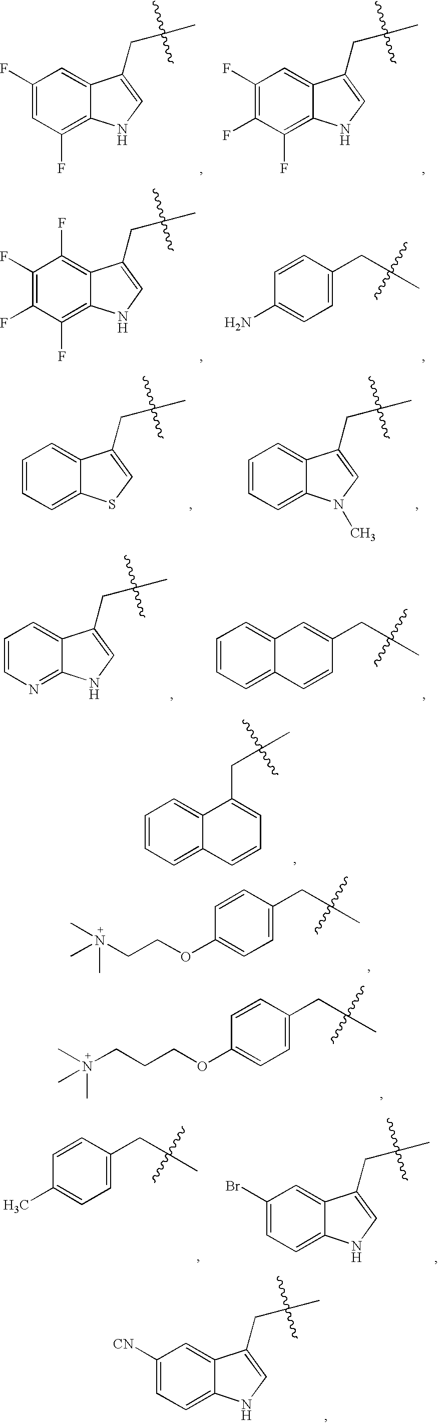

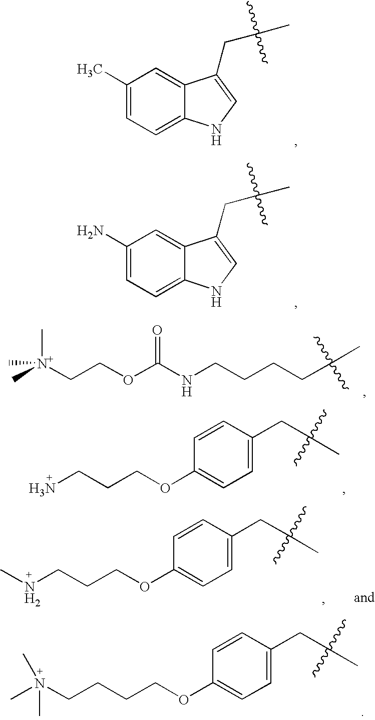

In another preferred embodiment, examples of unnatural amino acids for incorporation into mammalian cells also include, but are not limited to, those represented by the following Formula (II):

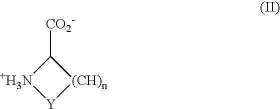

wherein: Y is CH₂, (CH)ₙ, N, O, or S, and n is 1 or 2. Examples of such compounds include, but are not limited to, the following compounds:

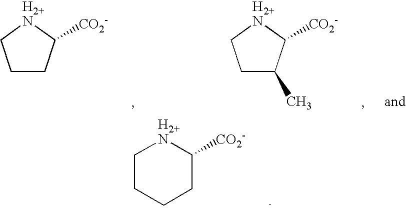

Note also that racemic amino acids can be used because only L-amino acids, and not D-amino acids, are incorporated. Cornish, et al. (1995) Angew. Chem. Int. Ed. Engl. 34: 621-633.

In a preferred embodiment, after synthesis of the relevant mRNA and acylated-tRNA, the species are co-injected into intact Xenopus oocytes such as those described in Nowak et al. (1998) Methods in Enzymol 293:515 using standard procedures known in the art. During translation the ribosome incorporates the unnatural amino acid into the nascent peptide at the position of the engineered stop codon, and an altered HERG channel is expressed on the oocyte membrane.

An electrophysiological method such as the current clamp or, preferably, the voltage clamp is used to assess the ligand-binding capabilities of altered ion channels or receptors. The current clamp assay measures ligand binding to a receptor or ion channel by detecting changes in the oocyte membrane potential associated with ion conduction across the cell membrane. The voltage clamp measures the voltage-clamp currents associated with ion conduction across the cell membrane. These currents vary with time, with the concentrations of agonists and antagonists, and with membrane potential, and these variations measure the number of open channels at any instant. Such electrophysiological methods are well known in the art (Hille, 2001; Methods in Enzymology, Vol 152) and have been used extensively for the study of ion channels in the Xenopus oocyte expression system.

Other ligand-binding assays can be developed to measure ligand binding events that do not involve changes in membrane potential. While one skilled in the art is capable of selecting a biochemical assay for use with a particular expression system, unnatural amino acid, ion channel, ligand, and modulator involved in a particular study, we describe here some example ligand-binding assays. The invention is not limited by the particular binding assay employed.

In one embodiment, a labeled ligand is used to physically detect the presence of the bound or unbound ligand. Various types of labels, including but not limited to radioactive, fluorescent, and enzymatic labels, have been used in binding studies and are well known in the art. Labeled ligands can be commercially obtained or prepared using techniques known in the art. A binding assay using a radioactively labeled ligand may include the following steps: (1) incubating purified ion channels or oocytes expressing ion channels with the labeled ligand, (2) allowing an appropriate time for ligand-binding, (3) counting the number of bound ligands using a scintillation counter, and (4) comparing the differences in radioactive counts for altered and unaltered channels.

Ion channel/ligand binding data are compiled to create a model of a ligand binding event. The contribution of specific amino acid side chains to ligand binding is inferred from the comparative properties of a natural amino acid with the substituted unnatural amino acid. Therefore, the production of meaningful data will depend in part on the selection of appropriate substitutions. While one skilled in the art is capable of selecting an unnatural amino acid substitution to investigate a putative channel/ligand interaction, we provide some examples of how relevant information is extrapolated from these experiments.

(1) A cation-p interaction is important if fluoro-, cyano-, and bromo- amino acid derivatives, substituted for natural aromatic amino acids, abrogate ligand binding. When incorporated into an aromatic amino acid, these substituents withdraw electron density from the aromatic ring, weakening the putative electrostatic interaction between a positively charged group on the ligand and the aromatic moiety. Fluoro- derivatives are often preferred because fluorine is a strong electron-withdrawing group, and often adds negligible steric perturbations.

(2) Hydrophobic interactions at a given position are important if ligand binding is affected by substitutions that increase hydrophobicity without significantly altering the sterics of the side chain, thereby allowing the importance of hydrophobic interactions to be investigated in the absence of artificial steric constraints. One example of such a manipulation is conversion of a polar oxygen to a nonpolar CH₂ group, as in O-Methyl-threonine to isoleucine. Other methods to increase hydrophobicity, such as increasing side chain length, as in the substitution of allo-isoleucine for valine, or β-branch addition, as in the substitution of norvaline for isoleucine, or γ-branch addition, as in the substitution of t-butylalanine for isoleucine, may produce results that support the importance of hydrophobic interactions.

(3) A local α-helix or β-sheet structure is important if an α-hydroxy acid substitution influences ligand binding. Incorporation of an α-hydroxy acid into the peptide backbone will produce an ester linkage instead of an amide bond. Since the amide hydrogen bond is important for stabilization of local α-helices and β-sheets, the α-hydroxy acid substitution disrupts these structures.

(4) By incorporating the phosphorylated or glycosylated analogue of a given amino acid into the ion channel, the investigator can compare ligand binding in the presence or absence of the putative modification.

(5) Using photoreactive unnatural amino acids, the importance of specific side chains or protein modifications can be studied. For example, addition of the photoremovable nitrobenzyl group to the side chain of an amino acid can prevent interactions with the ligand or block side chain modifications such as phosphorylation and methylation. UV irradiation removes the nitrobenzyl group thereby restoring the amino acid to its native form. Therefore, ligand-binding measurements taken before and after UV irradiation can uncover side chain contributions to ligand binding. Similarly, the importance of local protein structures such as loops can be investigated by incorporating the unnatural amino acid (2-nitrophenyl) glycine (Npg). Irradiation of the Npg-modified amino acid triggers proteolysis of the protein channel backbone. If UV irradiation disrupts ligand binding to the Npg-modified channel, a structure near the incorporated unnatural amino acid is likely important.

(6) Fluorescent reporter groups such as the nitrobenzoxadiazole (NBD) fluorophore or spin labels such as nitroxyl can be incorporated into the ion channel using unnatural amino acids containing these labels. For example, after incorporation of an NBD-amino acid into the channel, fluorescence resonance energy transfer between a fluorescently-labeled ligand and the NBD-amino acid can provide information such as the distance between the amino acid residue and the ligand-binding site.

Compounds of interest that will be screened for binding affinity to the modified HERG channel include, but are not limited to antiarrhythmic drugs. It is known that many structurally diverse compounds block HERG channels, therefore, any of these compounds are candidates for screening with the inventive system. Particular preferred compounds include MK-499, terfenadine, cisapride, and dofetilide.

The following examples are provided for illustration purposes, and are not intended to be limiting.

### EXAMPLES

Materials:

DNA oligonucleotides were synthesized on an Expedite DNA synthesizer (Perceptive Biosystems, Framingham, Mass.). Restrictions endonucleases and T4 ligase were purchased from New England Biolabs (Beverly, Mass.). T4 polynucleotide kinase, T4 DNA ligase, and Rnase inhibitor were purchased from Boehringer Mannheim Biochemicals (Indianapolis, Ind.). ³⁵S-methionine and ¹⁴C-labeled protein molecular weight markers were purchased from Amersham (Arlington Heights, Ill.). Inorganic pyrophosphatase is purchased from Sigma (St. Louis, Mo.). Stains-all is purchased from Aldrich (Milwaukee, Wis.). T7 RNA polymerase is either purified using the method of Grodberg and Dunn (1988) J. Bact. 170:1245 from the overproducing strain E. coli BL21 harboring the plasmid pAR1219 or purchased from Ambion (Austin, Tex.). For all buffers described, unless otherwise noted, final adjustment of pH is unnecessary.

Unnatural Amino Acids:

While most unnatural amino acids were purchased from commercial sources, other unnatural amino acids can be synthesized by known techniques. Tryptophan analogues were prepared using the method of Gilchrist et al. (1979) J. Chem. Soc. Chem. Commun. 1089-90. Tetrafluoroindole was prepared by the method of Rajh et al. (1979) Int. J. Pept. Protein Res. 14:68-79. 5, 7-Difluoroindole and 5,6,7-trifluoroindole were prepared by the reaction of CuI/dimethylformamide with the analogous 6-trimethylsilylacetylenylaniline.

Typically, the amino group is protected as the o-nitroveratryloxycarbonyl (NVOC) group, which is subsequently removed photochemically according to methods known in the art. However, for amino acids that have a photoreactive sidechain, an alternative, such as the 4-pentenoyl (4PO) group, a protecting group first described by Fraser-Reid, must be used. Madsen et al. (1995) J. Org. Chem. 60, 7920-7926; Lodder et al. (1997) J. Org. Chem. 62, 778-779. We present here a representative procedure based on the unnatural amino acid (2-nitrophenyl)glycine (Npg), as described in England, et al. Proc. Natl. Acad. Sci. USA (in press).

N-4PO-D,L-(2-nitrophenyl)glycine. The unnatural amino acid D,L-(2-nitrophenylglycine) hydrochloride was prepared according to Davis et al. (1973) J. Med. Chem. 16, 1043-1045; Muralidharan et al. (1995) J. Photochem. Photobiol. B: Biol. 27, 123-137. The amine was protected as the 4-pentenoyl (4PO) derivative as follows. To a room temperature solution of (2-nitrophenyl)glycine hydrochloride (82 mg, 0.35 mmol) in H₂O:dioxane (0.75 ml:0.5 ml) was added Na₂CO₃ (111 mg, 1.05 mmol), followed by a solution of 4-pentenoic anhydride (70.8 mg, 0.39 mmol) in dioxane (0.25 ml). After 3 hours the mixture was poured into saturated NaHSO₄ and extracted with CH₂Cl₂. The organic phase was dried over anhydrous Na₂SO₄ and concentrated in vacuo. The residual oil was purified by flash silica gel column chromatography to yield the title compound (73.2 mg, 75.2%) as a white solid. ¹H NMR (300 MHz, CD₃OD) δ 8.06 (dd, J=1.2, 8.1 Hz, 1H), 7.70 (ddd, J=1.2, 7.5, 7.5 Hz, 1H), 7.62-7.53 (m, 2H), 6.21 (s, 1H), 5.80 (m, 1H), 5.04-4.97 (m, 2H), 2.42-2.28 (m, 4H). HRMS calcd. for C₁₃H₁₄N₂O₅ 279.0981, found 279.0992.

N-4PO-D,L-(2-nitrophenyl)glycinate cyanomethyl ester. The acid was activated as the cyanomethyl ester using standard methods known in the art. (Robertson et al. (1989) Nucleic Acids Res. 17, 9649-9660; Ellman et al. (1991) Meth. Enzym. 202, 301-336. To a room temperature solution of the acid (63.2 mg, 0.23 mmol) in anhydrous DMF (1 ml) was added NEt₃ (95 μl, 0.68 mmol) followed by ClCH₂CN (1 ml). After 16 hours the mixture was diluted with Et₂O, and extracted against H₂O. The organic phase was washed with saturated NaCl, dried over anhydrous Na₂SO₄, and concentrated in vacuo. The residual oil was purified by flash silica gel column chromatography to yield the title compound (62.6 mg, 85.8%) as a yellow solid. ¹H NMR (300 MHz, CDCl₃) δ 8.18 (dd, J=1.2, 8.1 Hz, 1H), 7.74-7.65 (m, 2H), 7.58 (ddd, J=1.8, 7.2, 8.4 Hz, 1H), 6.84 (d, J=7.8 Hz, 1H), 6.17 (d, J=6.2 Hz, 1H), 5.76 (m, 1H), 5.00 (dd, J=1.5, 15.6 Hz, 1H), 4.96 (dd, J=1.5, 9.9 Hz, 1H), 4.79 (d, J=15.6 Hz, 1H), 4.72 (d, J=15.6 Hz, 1H), 2.45-2.25 (m, 4H). HRMS calcd. for C₁₆H₁₇N₃O₅ 317.1012, found 317.1004.

N-4PO-(2-nitrophenyl)glycine-dCA. The dinucleotide dCA was prepared as reported by Schultz (Id.) with the modifications described by Kearney et al. (1996) Mol. Pharmacol. 50, 1401-1412. The cyanomethyl ester was then coupled to dCA as follows. To a room temperature solution of dCA (tetrabutylammonium salt, 20 mg, 16.6 μmol) in anhydrous DMF (400 μl) under argon was added N-4PO-D,L-(2-nitrophenyl)glycinate cyanomethyl ester (16.3 mg, 51.4 μmol). The solution was stirred for 1 hour and then quenched with 25 mM NH₄OAc, pH 4.5 (20 μl). The crude product was purified by reverse-phase semi-preparative HPLC (Whatman Partisil 10 ODS-3 column, 9.4 mm×50 cm), using a gradient from 25 mM NH₄OAc, pH 4.5 to CH₃CN. The appropriate fractions were combined and lyophilized. The resulting solid was redissolved in 10 mM HOAc/CH₃CN and lyophilized to afford 4PO-Npg-dCA (3.9 mg, 8.8%) as a pale yellow solid. ESI-MS M⁻896 (31), [M−H]⁻895 (100), calcd for C₃₂H₃₆N₁₀O₁₇P₂ 896. The material was quantified by UV absorption (ε₂₆₀˜37,000 M⁻¹ cm⁻¹).

Suppressor tRNA Design and Synthesis:

Suppressor tRNA which encode for the desired unnatural amino acid were made, for example, by the methods taught in Nowak et al. (1998) and Petersson et al. (2002) RNA 8(4):542-7. The following procedure was followed for the suppressor tRNA THG73. The gene for T. thermophila tRNA^{Gln} CUA G73, flanked by an upstream T7 promoter and a downstream Fok I restriction site, and lacking CA at positions 75 and 76, was constructed from eight overlapping DNA oligonucleotides (SEQ ID NOs: 1-8), shown below, and cloned into the pUC19 vector.

SEQ ID NO:1
5′-AATTCGTAATACGACTCACTATAGGTTCTATAG-3′
SEQ ID NO:2
3′-GCATTATGCTGAGTGATATCCAAGA-5′
SEQ ID NO:3
5′-TATAGCGGTTAGTACTGGGGACTCTAAA-3′
SEQ ID NO:4
3′-TATCATATCGCCAATCATGACCCCTGAG-5′
SEQ ID NO:5
5′-TCCCTTGACCTGGGTTCG-3′
SEQ ID NO:6
3′-ATTTAGGGAACTGGACCC-5′
SEQ ID NO:7
5′-AATCCCAGTAGGACCGCCATGAGACCCATCCG-3′
SEQ ID NO:8
3′-AGCTTAGGGTCATCCTGGCGGTACTCTGGGTAGGCCTAG-5′

Digestion of the resulting plasmid (pTHG73) with Fok I gave a linearized DNA template corresponding to the tRNA transcript, minus the CA at positions 75 and 76. In vitro transcription of Fok I linearized pTHG73 was done as described by Sampson et al. (1988) Proc. Natl. Acad. Sci. 85:1033. The 74-nucleotide tRNA transcript, tRNA- THG73 (minus CA), was purified to single nucleotide resolution by denaturing polyacrylamide electrophoresis and then quantitated by ultraviolet absorption.

Chemical Acylation of tRNAs and Removal of Protecting Groups:

The a-NH₂— protected dCA-amino acids or dCA were enzymatically coupled to the THG73 FokI runoff transcripts using T4 RNA ligase to form a full-length chemically charged a-NH₂— protected aminoacyl-THG73 or a full-length but unacylated THG73-dCA.

Prior to ligation, 10 μl of THG73 (1 μg/μl in water) was mixed with 5 μl of 10 mM HEPES, pH 7.5. This tRNA/HEPES premix was heated at 95° C. for 3 min and allowed to cool slowly to 37° C.

After incubation at 37° C. for 2 hours, DEPC-H₂O (52 μl) and 3M sodium acetate, pH 5.0 (8 μl), were added and the reaction mixture was extracted once with an equal volume of phenol (saturated with 300 mM sodium acetate, pH 5.0):CHCl₃: isoamyl alcohol (25:24:1) and once with an equal volume of CHCl₃:isoamyl alcohol (24:1), then precipitated with 2.5 volumes of cold ethanol at −20° C. The mixture was centrifuged at 14,000 rpm at 4° C. for 15 min, and the pellet was washed with cold 70% (v/v) ethanol, dried under vacuum, and resuspended in 7 μl 1 mM sodium acetate, pH 5.0. The amount of a-NH₂— protected amino acyl-THG73 was quantified by measuring A₂₆₀, and the concentration was adjusted to 1 μg/μl with 1 mM sodium acetate pH 5.0.

The ligation efficiency was determined from analytical PAGE. The a-NH₂— protected amino acyl-tRNA partially hydrolyzes under typical gel conditions, leading to multiple bands, so the ligated tRNA was deprotected prior to loading. Such deprotected tRNAs immediately hydrolyze on loading. Typically, 1 μg of ligated tRNA in 10 μl BPB/XC buffer was loaded onto the gel, and 1 μg of unligated tRNA was run as a size standard. The ligation efficiency was determined from the relative intensities of the bands corresponding to ligated tRNA (76 bases) and unligated tRNA (74 bases).

Generation of mRNA:

The mRNA was synthesized in vitro from a mutated complementary cDNA clone containing a stop codon, TAG, at the amino acid position of interest. For the nonsense codon suppression method, it is desirable to have the gene of interest in a high-expression plasmid, so that functional responses in oocytes may be observed 1-2 days after injection. Among other considerations, this minimizes the likelihood of reacylation of the suppressor tRNA. Although there are many high-expression oocyte plasmids available to one of skill in the art, we describe here the high-expression plasmid pAMV-PA, generated by modifying the multiple cloning region of pBluescript SK⁺ . Nowak et al. (1998) Methods in Enzymol. 293:515.

At the 5′ end, an alfalfa mosaic virus (AMV) sequence was inserted, and at the 3′ end a poly(A) tail was added, providing the plasmid pAMV-PA. mRNA transcripts containing the AMV region bind the ribosomal complex with high affinity, leading to 30 fold increase in protein synthesis. Including a 3′poly (A) tail was shown to increase mRNA half-life, therefore increasing the amount of protein synthesized. The gene of interest was subcloned into pAMV-PA such that the AMV region is immediately 5′ of the ATG start codon of the gene (i.e. the 5′ untranslated region of the gene was completely removed). The plasmid pAMV-PA was made available from C. Labaraca at Caltech.

TAG stop codons at positions where unnatural amino acid incorporation is desired were produced by site directed mutagenesis. Suitable site-directed mutagenesis methods used to create stop codons at the desired positions include the Transformer kit (Clontech, Palo Alto, Calif.), the Altered Sites kit (Stratagene, La Jolla, Calif.), and standard polymerase chain reaction (PCR) cassette mutagenesis procedures. With the first two methods, a small region of the mutant plasmid (400-600 base pairs) was subcloned into the original plasmid. With all methods, the inserted DNA regions were checked by automated sequencing over the ligated sites. The pAMV-PA plasmid constructs were linearized with NotI, and mRNA transcripts were generated using the mMessage mMachine T7 RNA polymerase kit (Ambion, Austin, Tex.).

Oocytes—Preparation and Injection:

Oocytes were removed from Xenopus laevis using techniques known in the art. Quick, M., Lester, H. A. (1994). Methods for expression of excitability proteins in Xenopus Oocytes. In Ion Channels of Excitable Cells. (Narahashi, T., ed.), pp 261-279, Academic Press, San Diego, Calif., USA. Oocytes were maintained at 18° C. in ND96 solution consisting of 96 mM NaCl, 2 mM KCl, 1 mM MgCl₂, 1.8 mM CaCl₂, and 5 mM HEPES (pH 7.5), supplemented with sodium pyruvate (2.5 mM), gentamicin (50 μg/ml), theophylline (0.6 mM) and horse serum (5%). Prior to injection, the NVOC-aminoacyl-tRNA (1 μg/μl) in 1 mM NaOAc (pH 5.0) was deprotected by irradiating for 5 min with a 1000 W xenon arc lamp (Oriel) operating at 600 W equipped with WG-335 and UG-11 filters (Schott). The deprotected aminoacyl-tRNA was mixed 1:1 with a water solution of the desired mRNA. Oocytes were injected with 50 nl of a mixture containing 25-50 ng aminoacyl-tRNA and 12.5-18 ng of total ion channel mRNA (ratio of 20:1:1:1 for α:β:δ subunits).

Electrophysiology:

Two-electrode voltage-clamp recordings were performed 24 to 36 hours after injection using a GeneClamp500 circuit and a Digidata 1200 digitizer from Axon Instruments, Inc. (Foster City, Calif.) interfaced with a PC running pCLAMP6 or CLAMPEX software from Axon. The recording solution contained 96 mM NaCl, 2 mM MgCl₂, and 5 mM HEPES (pH 7.4). Whole-cell current responses to various ligand concentrations at indicated holding potentials (typically −60 mV) were fitted to the Hill equation, I/Iₘₐₓ=1/{1+(EC₅₀/[A])ⁿ}, where I is agonist-induced current at [A], Iₘₐₓ is the maximum current, EC₅₀ is the concentration inducing half-maximum response, and n is the Hill coefficient.

Development of Receptophore Model:

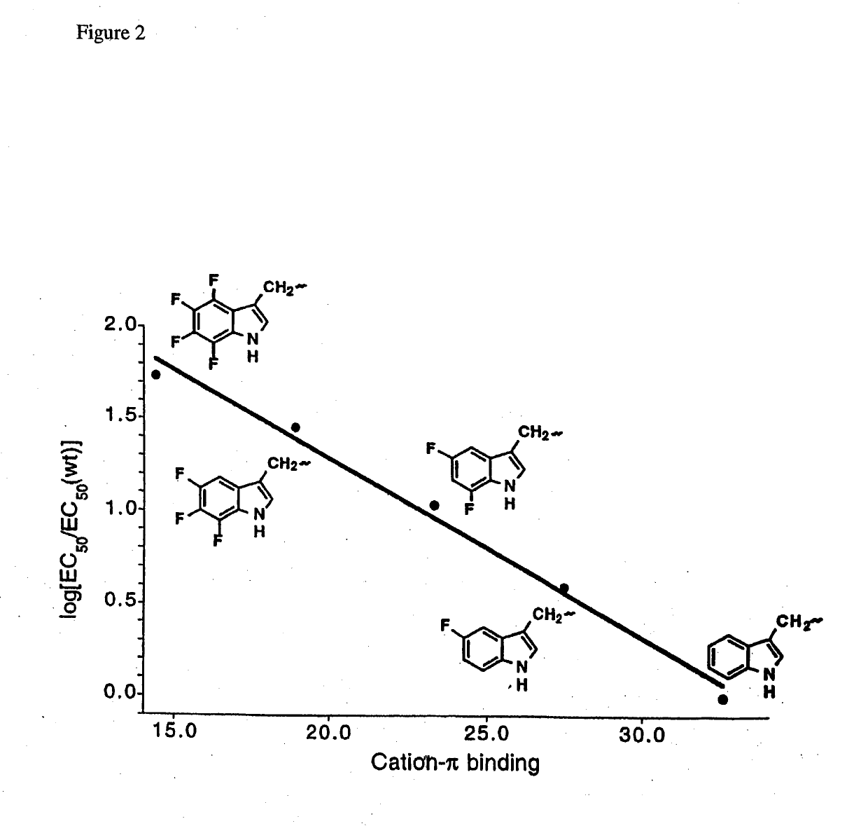

Dose-response curves were fitted to the Hill equation for the unaltered receptor (WT) and for unnatural amino acid substitutions at α-Trp 149. Substitutions include 5-F-Trp, 5,7-F₂-Trp, 5,6,7-F₃-Trp, and 4,5,6,7-F₄-Trp. The log[EC₅₀/EC_{50(WT)}] each substitution and for the unaltered receptor was plotted vs. cation-π binding ability of each fluorinated trp derivative. Cation-π binding ability for both trp and the fluorinated derivatives was predicted using ab initio quantum mechanical calculations. Mecozzi et al. (1996) J. Amer. Chem. Soc. 118: 2307-2308; Mecozzi et al. (1996) Proc. Natl. Acad. Sci. USA 93:10566-10571. Data fit the line y=3.2-0.096x, with a correlation coefficient r=0.99. See FIG. 2. These data are consistent with a cation-π bond between α-trp 149 and the quaternary ammonium of acetylcholine in the bound position because each substitution's EC₅₀ value corresponds well with the predicted loss in binding energy due to the substitution. After further systematic mapping of contacts between acetylcholine and the nicotinic acetylcholine receptor, a receptophore model describing the complete steric and electronic features involved in this interaction can be made.

Characterization of the Cation-π Interaction Site at Y652 and F656 Using Dofetilide:

This experiment characterizes the binding and electrophysiology of dofetilide and several of its analogues with the HERG channel and several of its mutants containing unnatural amino acid mutations at the Y652 and F656 sites to generate a detailed picture of the binding at this site. The dofetilide analogues were chosen to represent a range of binding affinities to the HERG channel. This experiment provides a range of interactions that allow for the definition of the pharmacophore for dofetilide binding to the HERG channel. The unnatural HERG channel mutants reveal details of the binding interactions that provide indications of the orientations of dofetilide and its analogues at the binding site. The dofetilide and dofetilide analogues used in this experiment, shown below, are known in the art and described in, for example, U.S. Pat. No. 4,959,366 and EP 649,838.

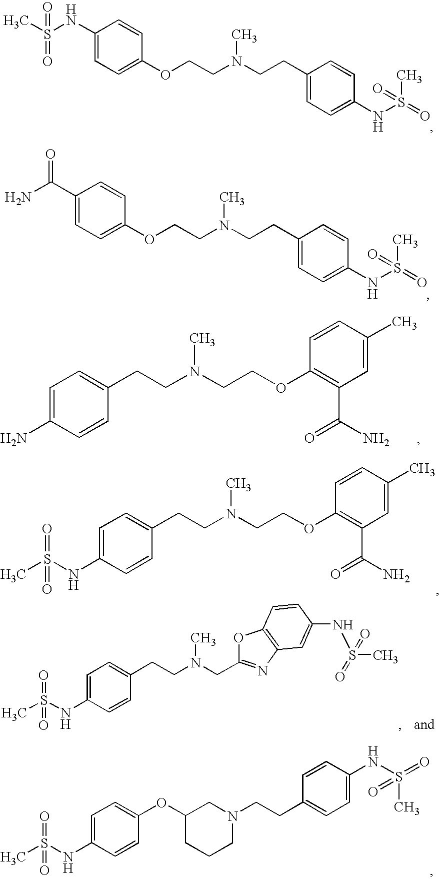

Construction of Concatenated Gene for HERG Channel:

A concatenated gene for the HERG channel will be constructed to allow delineation of the face of the channel to which compounds bind. There are four identical faces of the HERG channel, so a concatenated gene will allow determination of which specific face of the channel contains an interaction point. Those skilled in the art will proceed along the lines of the papers by, for example, Silverman et al. (J Biol Chem 1996 Nov. 29;271(48):30524-8) and Pessia et al. (EMBO J 1996 Jun. 17;15(12):2980-7).

All references cited herein are incorporated by reference in their entirety.

While the invention has been described in conjunction with examples thereof, it is to be understood that the foregoing description is exemplary and explanatory in nature, and is intended to illustrate the invention and its preferred embodiments. Through routine experimentation, the artisan will recognize apparent modifications and variations that may be made without departing from the spirit of the invention. Thus, the invention is intended to be defined not by the above description, but by the following claims and their equivalents.

We claim:

1. A method of incorporating unnatural amino acids into the HERG ion channel comprising:
a) determining sites of potential antagonist or agonist interaction with the HERG ion channel;
b) using the nonsense codon suppression method to incorporate unnatural amino acids into the sites determined in (a).

2. The method of claim 1 wherein said unnatural amino acids are represented by the Formula (I): 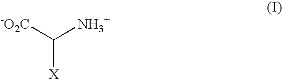
where X is selected from the group consisting of:

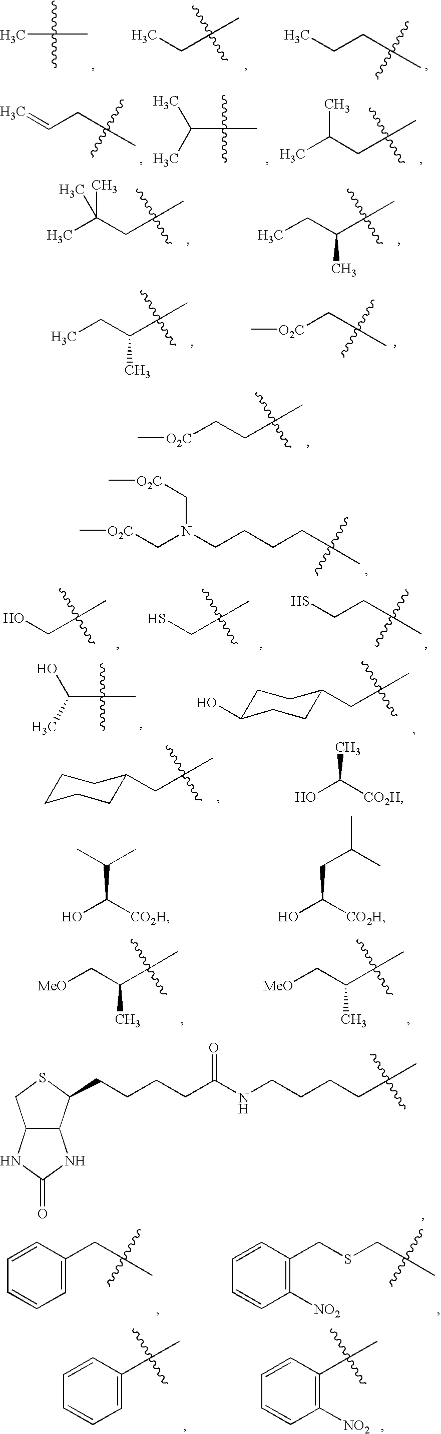

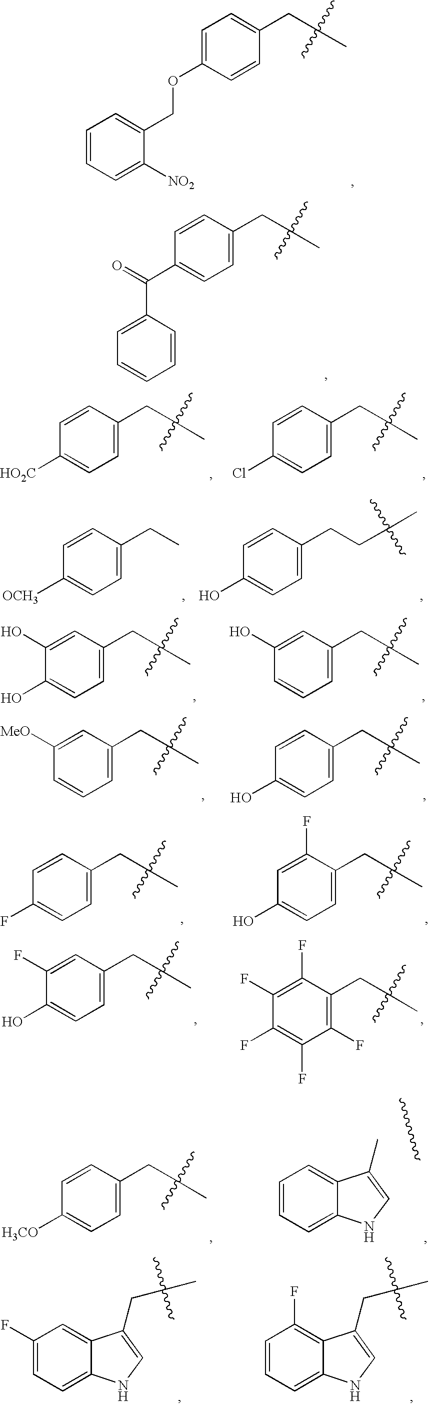

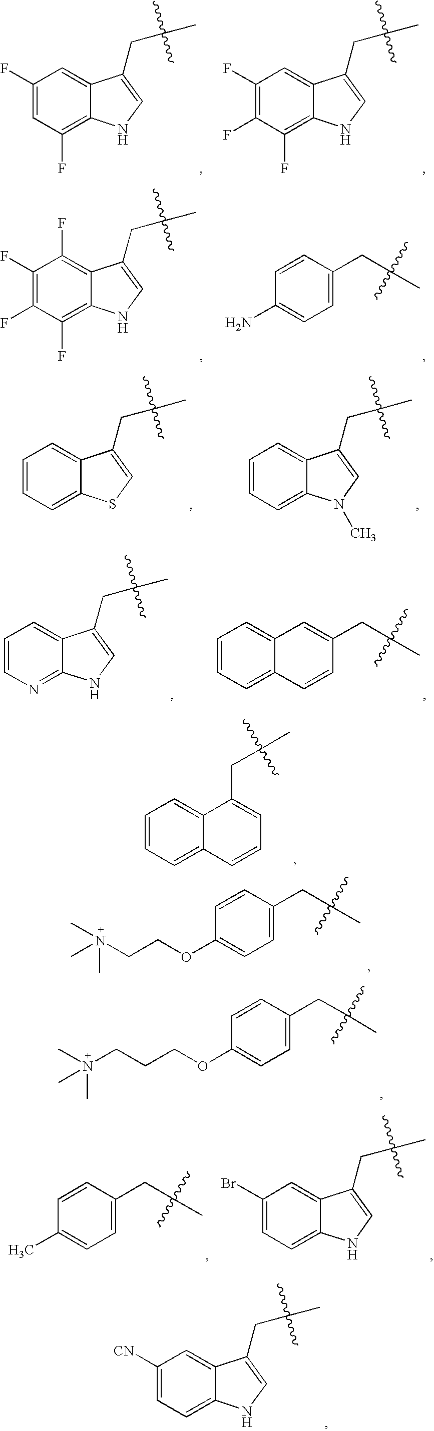

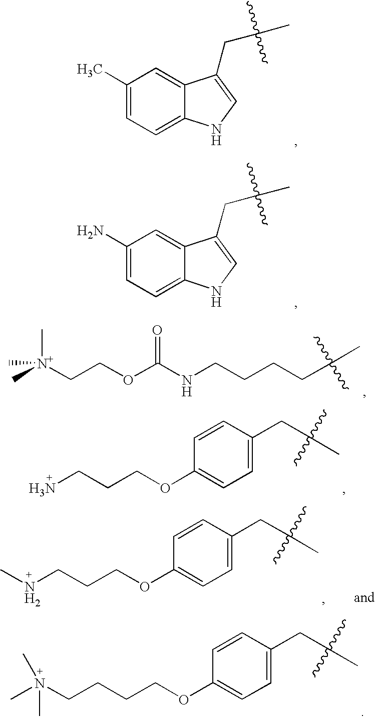

3. The method of claim 1 wherein said unnatural amino acids are represented by the Formula (II): 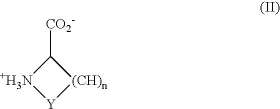
wherein: Y is CH₂, (CH)ₙ, N, O, or S, and n is 1 or 2.

4. The method of claim 1 wherein said unnatural amino acids are selected from the group consisting of:

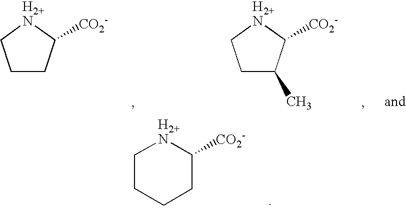

5. The method of claim 1 wherein the HERG ion channel is expressed in Xenopus oocytes.

6. A method of determining the nature of a compound's interaction with HERG comprising:
a) incorporating unnatural amino acids into binding and regulatory sites of HERG, resulting in an altered HERG;
b) measuring the compound's ability to bind to the altered HERG; and
c) comparing the results of step (b) to the same compound's ability to bind to an unaltered HERG.

7. A method of screening for compounds which cause cardiac toxicity comprising:
a) developing a screening assay system, wherein said system allows for searching of compounds that prolong QT interval on ECG readings;
b) using said screening assay system to determine details of the nature and location of HERG binding of said compounds;
c) predicting which compounds are causing said toxicity by evaluating how and where said compound binds to HERG.

8. A HERG screening assay system comprising a HERG ion channel which has been modified to replace native amino acids with unnatural amino acids, wherein the ion channel is expressed in vivo and compounds are screened for HERG binding affinity.

9. The screening assay system of claim 8 wherein the native amino acids to be replaced are selected from the group consisting of G648, Y652, F656, T623, and V625.

10. The screening assay system of claim 8 wherein said unnatural amino acids are represented by the Formula (I): 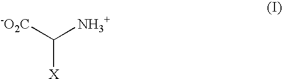
where X is selected from the group consisting of:

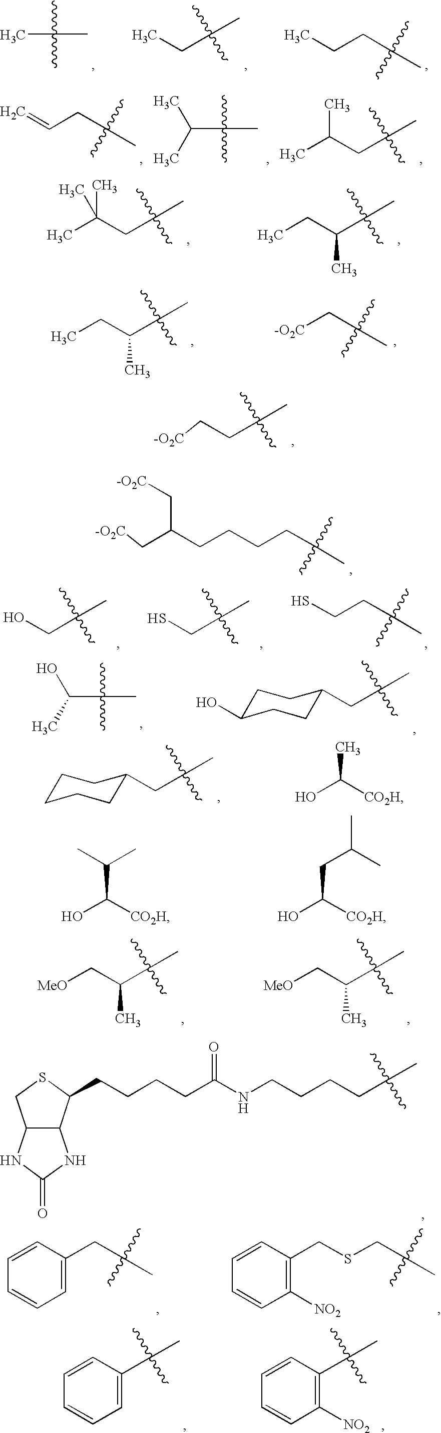

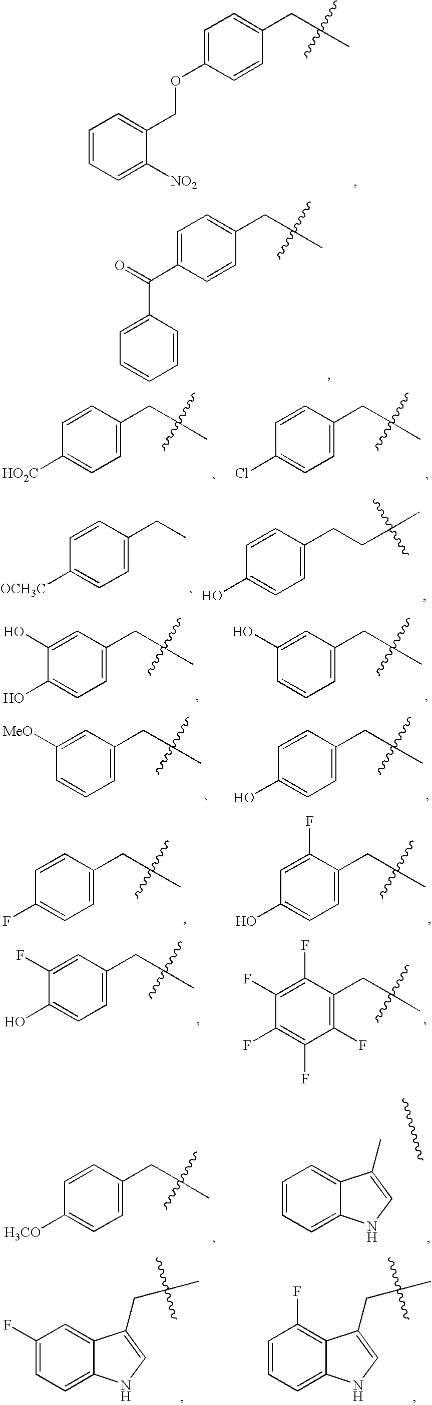

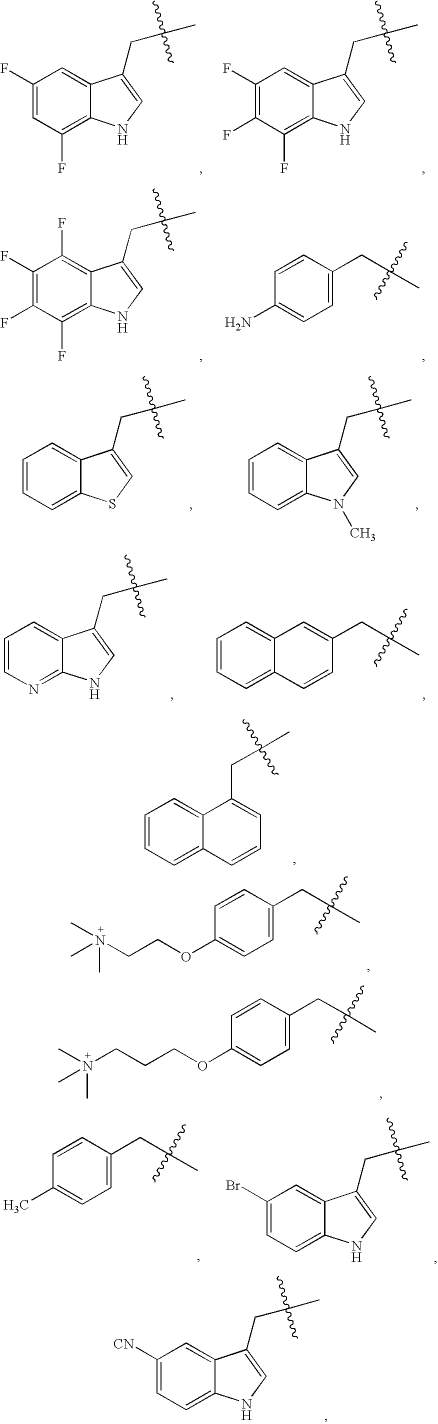

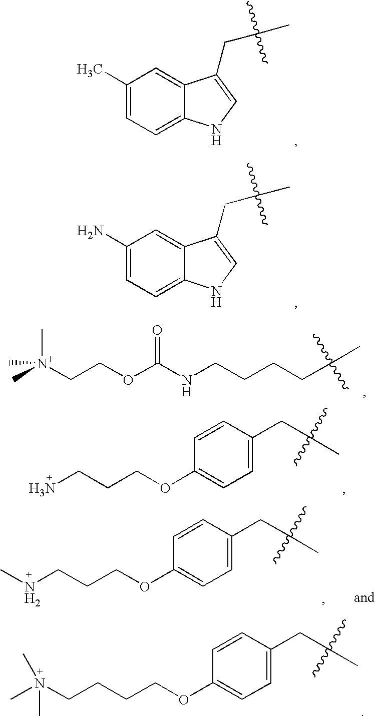

11. The screening assay system of claim 8 wherein said unnatural amino acids are represented by the Formula (II): 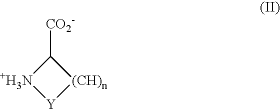
wherein: Y is CH₂, (CH)ₙ, N, O, or S, and n is 1 or 2.

12. The screening assay system of claim 8 wherein said unnatural amino acids are selected from the group consisting of:

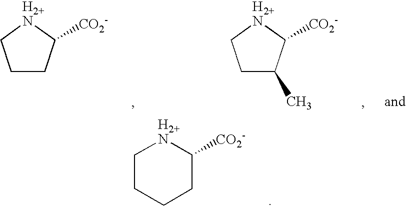

13. The screening assay system of claim 8 wherein said compounds to be screened for HERG binding affinity are dofetilide and dofetilide analogues.

14. The screening assay system of claim 13 wherein the dofetilide analogues are selected from the group consisting of: [chemical structure]
